(19) United States
(12) Patent Application Publication
(10) Pub. No.: US 2021/0369397 A1
(43) Pub. Date: Dec. 2, 2021

(54) DEVICE AND METHOD TO PROTECT MEDICAL PROFESSIONALS FROM PATHOGEN-CONTAINING DROPLETS WHILE ATTENDING TO PATIENTS

(71) Applicants: Robbie Slim, Fontana, CA (US); Christian Franco, Lake Elsinore, CA (US)

(72) Inventors: Robbie Slim, Fontana, CA (US); Christian Franco, Lake Elsinore, CA (US)

(21) Appl. No.: 16/884,606

(22) Filed: May 27, 2020

(51) Int. Cl. A61B 90/40

(52) U.S. Cl. CPC A61B 90/40; A61B 2560/04

(57) ABSTRACT

A shield and method for using the shield to protect medical professionals from pathogen-containing droplets that are being released from a patient's respiratory system while the patient is resting on a gurney mattress. The shield includes a shield frame. An attachment structure is connected to the shield frame. The attachment structure is for connecting the shield frame to the gurney mattress. A transparent cover is draped over the shield frame and is for preventing patient generated pathogen-containing droplets from being inhaled by medical professionals. The transparent covering has access holes for allowing medical professionals to access the patient.

The present invention relates to medical devices, and in particular, to medical devices used to protect medical professionals from pathogen-containing droplets.

### BACKGROUND

Intubation is a known medical procedure that can be dangerous for medical professionals performing the procedure. Intubation involves the introduction of a tube—the endotracheal tube—into the mouth and subsequently into the airway to help ventilate a patient during respiratory failure. Patients require this procedure when breathing autonomously becomes impossible.

During intubation, small particles from the patient's mouth and airway can become aerosolized, putting the medical professionals surrounding the patient during the procedure at an increased risk of contracting the disease.

Recently, the rapid spread of a virus (SARS-CoV-2) that causes Corona Virus Disease (COVID-19) has put medical professionals needing to intubate at an even higher risk due to multiple factors:
1. Respiratory distress and failure are common reasons for hospitalization for those that contract the Coronavirus, symptoms that may require intubation
2. There are no approved treatments, vaccines, or cures for COVID-19
3. The increased demand for masks due to laws in various countries mandating masks for the general public has led to a shortage of N95 and P100 masks typically used by medical professionals, leaving them with less protection.

What is needed is a lightweight, removable barrier that can be used to contain the spread of virus-containing droplets without inhibiting the medical professional in their execution of necessary ventilation procedures.

### SUMMARY OF THE INVENTION

The present invention provides a shield and method for using the shield to protect medical professionals from pathogen-containing droplets that are being released from a patient's respiratory system while the patient is resting on a gurney mattress. The shield includes a shield frame. An attachment structure is connected to the shield frame. The attachment structure is for connecting the shield frame to the gurney mattress. A transparent cover is draped over the shield frame and is for preventing patient generated pathogen-containing droplets from being inhaled by medical professionals. The transparent covering has access holes for allowing medical professionals to access the patient.

### BRIEF DESCRIPTION OF THE DRAWINGS

FIG. 1 is a perspective right-side view of a preferred embodiment of the present invention,

FIG. 2 is a perspective view of the structure taken from above, showing all attachment points of the structure to itself.

FIG. 2B shows a preferred method for connecting a transparent plastic sheet to the shield frame.

FIG. 3 is a perspective right-side view of the preferred embodiment of the present invention in use.

FIG. 4 shows a perspective view of a preferred shield frame

FIG. 5 shows a perspective view of a preferred shield frame attached to a gurney mattress.

### DETAILED DESCRIPTION OF THE PREFERRED EMBODIMENTS

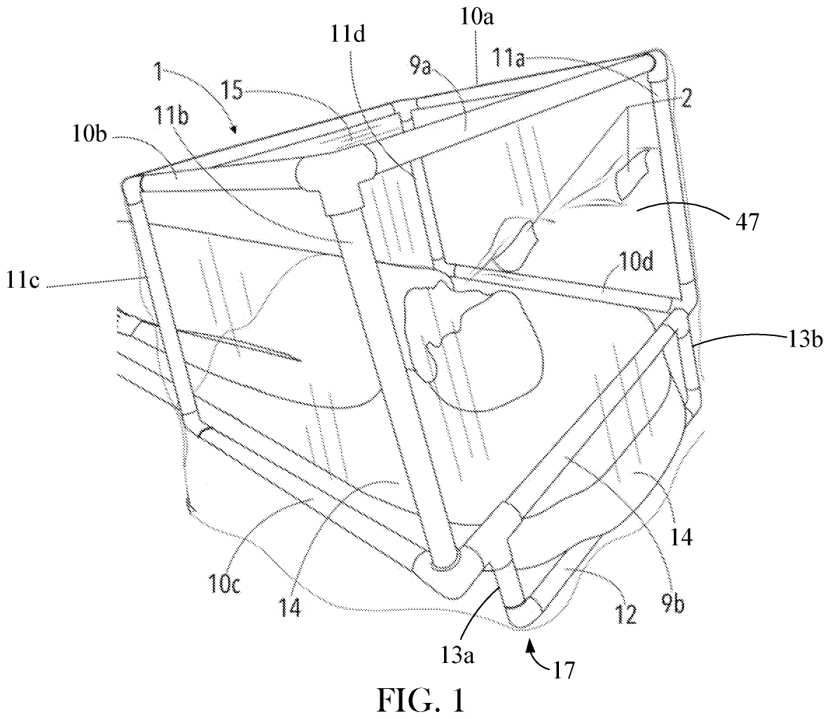

FIG. 1 shows a preferred embodiment of the present invention. Shield 1 is utilized to protect medical professionals from pathogen-containing droplets being released from a patient's respiratory system during intubation. Shield 1 includes shield frame 16. Shield frame 16 is fabricated from rigid polyvinyl chloride (PVC) piping and surrounds the patient on their left side, head, and right side—from the torso, up. Shield frame 16 is secured at the top, near the patient's head, by inserting the top of gurney mattress 14 through attachment piece 17. The patient's weight on the gurney mattress holds shield 1 in place. Shield frame 16 is covered by flexible plastic sheet 15 which serves to contain infectious aerosolized particles within the structure and prevent spread of infection to the medical staff administering the intubation. Plastic sheet 16 has two openings near the head of the patient so that a medical professional may insert his hands and forearms into the structure and perform the intubation without significant limitation. The structure is lightweight, made of only PVC and flexible plastic, and can therefore be removed following intubation (or other ventilation procedures) when the risk to medical staff is lessened.

### Fabrication of the Shield

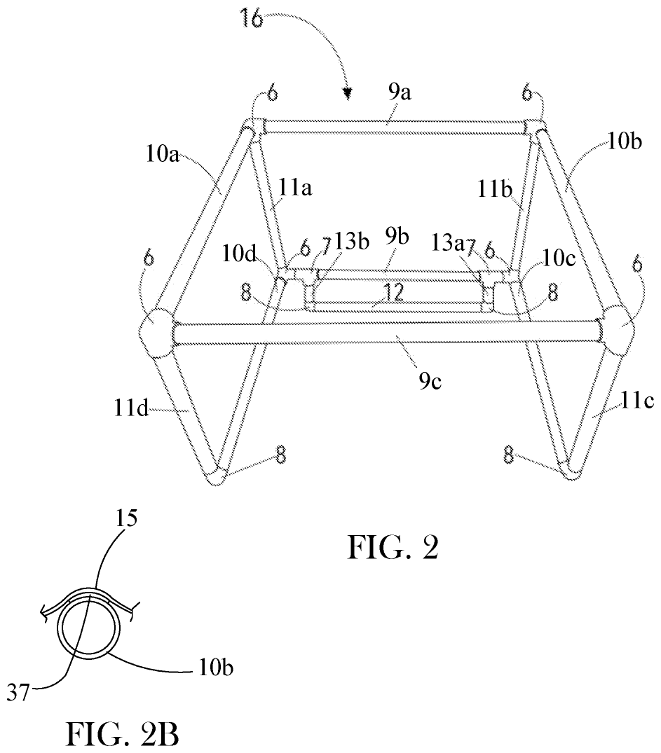

Shield frame 16 is preferably fabricated from 1½-inch diameter PVC pipe (FIG. 2). Shield frame 16 includes three front straight sections 9a-9c, four side straight sections 10a-10d, lower straight section 12, straight lower side pieces 13a-13b, four vertical straight sections 11a-11d, four two-way elbow sections 8, six three-way elbow sections 6, and two T-sections 7. In a preferred embodiment the PVC sections of shield frame 16 are joined in the manner as shown using PVC glue for rigid connections. By making the connections shown, shield frame 16 has been formed to include a four-sided top frame (bordered by straight sections 9a, 10b, 9c and 10a), a four-sided side frame (bordered by straight sections 10a, 11a, 10d and 11d), a four-sided side frame (bordered by straight sections 10b, 11c, 10c and 11b), a four-sided front frame (bordered by straight sections 9a, 11b, 9b, and 11a), a three-sided rear frame (bordered by straight sections 9c, 11c and 11d), a three-sided bottom frame (bordered by straight sections 9b, 10d, and 10c), and four-sided attachment structure 17 (bordered by straight sections 9b, 13b, 13a, and 12).

In one embodiment of the present invention, the dimensions of the preferred embodiment are length (L)=29″, width (W)=27″, height (H)=22″ with the width (W) of attachment piece 17=22½ ″ and the height (H) of the attachment piece 17=6½″. It should be understood that these specific dimensions of shield frame 16 may be easily modified based on the needs of the medical personnel and patient. For example, in another preferred embodiment shield 1 is utilized during breathing treatments to open up the airway of an asthmatic patient during an asthmatic attack. In this preferred embodiment shield 1 utilizes identical construction with smaller dimensions, i.e., length (L)=28″, width (W)=19″, height (H)=22″. Likewise, the dimensions of attachment piece 17 may be modified accordingly.

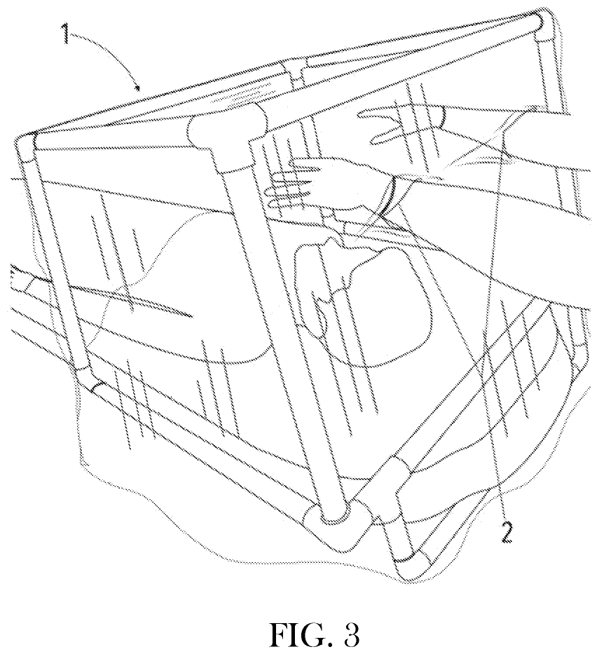

Prior to use, shield frame 16 is covered with thin, flexible, see-through plastic sheet 15 (FIG. 1) which is preferably secured by double-sided tape 37 (FIG. 2B). As shown clearly in FIGS. 1 and 3, access holes 2 are cut out of plastic cover sheet 15 at front wall 47 of shield 1 so as to allow easy access to the patient's head.

### Utilization of the Shield

In FIG. 1, a patient is shown resting on gurney mattress 14. Shield 1 has been placed on top of the gurney mattress and is covering the patient. Lower right arm 10d is on the right side of the patient and along the right side of gurney mattress 14. Lower left arm 10c has been placed on the left side of the patient and is along the left side of gurney mattress 14, as shown. Straight section 9b of attachment structure 17 is resting on the head of gurney mattress 14 and is above the head of the patient.

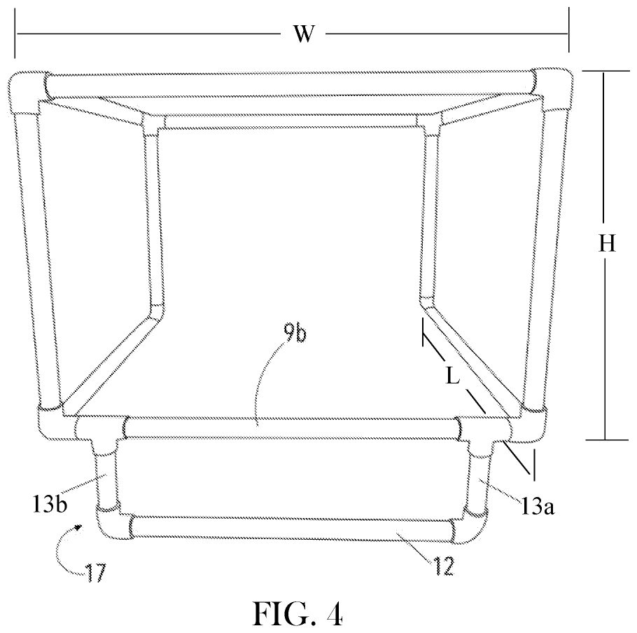

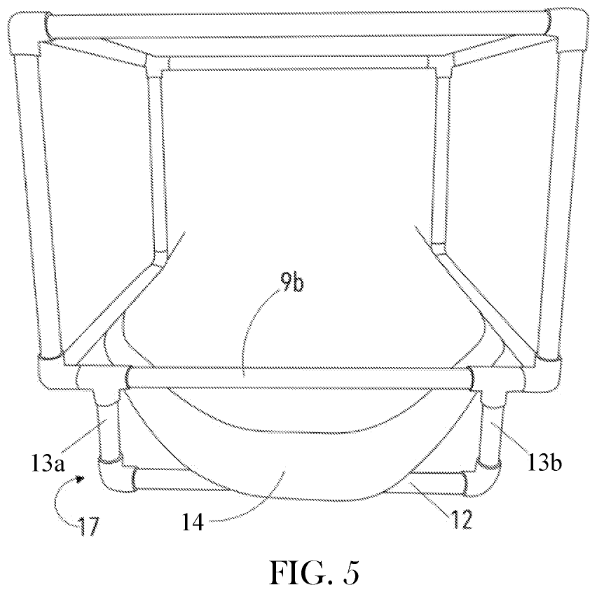

Shield 1 is secured to gurney mattress 14 by placing the top of gurney mattress 14 through attachment structure 17 (see also FIGS. 4-5). Attachment structure 17 is formed by straight section 9b, lower left-side piece 13a, lower right-side piece 13b, and underside piece 12. Flexible plastic cover sheet 15 is draped over the shield frame 16 (FIG. 1) and over and around the patient. Holes 2 have been cut out of plastic cover sheet 15 and positioned on front wall 47 of shield 1 so as to allow easy access to the patient's head. The utilization of holes 2 is depicted in FIG. 3. For example, in FIG. 3 a medical professional has inserted his hands and part of his forearms into holes 2 to perform an intubation with limited inhibition and limited risk of infection.

Although the above-preferred embodiments have been described with specificity, persons skilled in this art will recognize that many changes to the specific embodiments disclosed above could be made without departing from the spirit of the invention. Therefore, the attached claims and their legal equivalents should determine the scope of the invention.

What is claimed is:

1. A shield for protecting medical professionals from pathogen-containing droplets that are being released from a patient's respiratory system while the patient is resting on a gurney mattress, said shield comprising:
A. a shield frame,
B. an attachment structure connected to said shield frame, said attachment structure for connecting said shield frame to said gurney mattress,
C. a transparent covering draped over said shield frame, said transparent covering for preventing patient generated pathogen-containing droplets from being inhaled by medical professionals, said transparent covering comprising at least one access hole for allowing medical professionals to access the patient.

2. The shield as in claim 1 wherein said shield frame is fabricated from PVC piping.

3. The shield as in claim 1 wherein said shield frame comprises:
A. a four-sided top frame,
B. two four-sided side frames connected to said four-sided top frame,
C. a four-sided front frame, connected to said two four-sided side frames, said four-sided top frame, and said attachment structure,
D. a three-sided rear frame, connected to said four-sided top frame and said two four-sided side frames, and
E. a three-sided bottom frame, connected to said three-sided rear frame and said two-four-sided side frames.

4. The shield as in claim 1 wherein said gurney mattress is inserted through said attachment structure and wherein said shield is secured by the weight of said gurney mattress and the weight of the patient.

5. The shield as in claim 1 wherein said transparent covering is a thin, flexible plastic sheet.

6. The shield as in claim 1, further comprising double sided tape for connecting said transparent covering to said shield frame.

7. The shield as in claim 1, wherein said at least one access holes is two access holes.

8. A method for protecting medical professionals from pathogen-containing droplets being released from a patient's respiratory system while the patient is resting on a gurney mattress, said method comprising the steps of:
A. placing a patient on a gurney mattress,
B. placing a shield over the patient on said gurney mattress, said shield comprising:
i. a shield frame,
ii. an attachment structure connected to said shield frame, said attachment structure for connecting said shield frame to said gurney mattress, and
iii. a transparent covering draped over said shield frame, said transparent covering for preventing patient generated pathogen-containing droplets from being inhaled by medical professionals, said transparent covering comprising at least one access hole for allowing medical professionals to access the patient,
C. inserting the top of said gurney mattress through said attachment structure, and
D. reaching through said at least one access hole to access the patient.

9. The method as in claim 8 wherein said shield frame is fabricated from PVC piping.

10. The method as in claim 8 wherein said shield frame comprises:
A. a four-sided top frame,
B. two four-sided side frames connected to said four-sided top frame,
C. a four-sided front frame, connected to said two four-sided side frames, said four-sided top frame, and said attachment structure,
D. a three-sided rear frame, connected to said four-sided top frame and said two four-sided side frames, and
E. a three-sided bottom frame, connected to said three-sided rear frame and said two-four-sided side frames.

11. The method as in claim 8 wherein said gurney mattress is inserted through said attachment structure and wherein said shield is secured by the weight of said gurney mattress and the weight of the patient.

12. The method as in claim 8 wherein said transparent covering is a thin, flexible plastic sheet.

13. The method as in claim 8, further comprising double sided tape for connecting said transparent covering to said shield frame.

14. The method as in claim 8, wherein said at least one access holes is two access holes.
